(19) United States
(12) Patent Application Publication
(10) Pub. No.: US 2011/0200635 A1
(43) Pub. Date: Aug. 18, 2011

(54) COMBINED INFLUENZA VACCINES FOR SEASONAL AND PANDEMIC PROTECTION

(75) Inventors: Angelika Banzhoff, Marburg (DE); Ralf Leo Clemens, Siena (IT)

(73) Assignee: NOVARTIS AG

(21) Appl. No.: 12/997,577

(22) PCT Filed: Jun. 12, 2009
(86) PCT No.: PCT/IB09/06038

§ 371 Date: Apr. 22, 2011

Related U.S. Application Data
(60) Provisional application No. 61/131,918, filed on Jun. 12, 2008.

(30) Foreign Application Priority Data
Mar. 31, 2009 (GB) 0905570.8

(51) Int. Cl. A61K 39/145; A61J 1/14; A61M 5/31

(52) U.S. Cl. 424/206.1; 604/403; 604/82

(57) ABSTRACT

Current approaches to influenza vaccination focus either on seasonal strains or pandemic strains. Current seasonal vaccines typically include antigens from two influenza A strains (H1N1 and H3N2) and one influenza B strain. Current pandemic vaccines focus on H5N1 influenza A virus strains. It is an object of the invention to provide further and improved ways of preparing vaccines that can raise immunity against both seasonal and pandemic strains.

This application claims the benefit of U.S. provisional application 61/131,918, filed Jun. 12, 2008, and United Kingdom patent application 0905570.8, filed Mar. 31,2009, the complete contents of both of which are incorporated herein by reference.

### TECHNICAL FIELD

This invention is in the field of vaccines for protecting against influenza virus infection, and in particular vaccines that include antigens derived from current seasonal strains and pandemic strains

### BACKGROUND ART

Current approaches to influenza vaccination focus either on seasonal strains or pandemic strains. Current seasonal vaccines typically include antigens from two influenza A strains (H1N1 and H3N2) and one influenza B strain. Current pandemic vaccines focus on H5N1 influenza A virus strains.

Vaccines that can raise immunity against both seasonal and pandemic strains are disclosed in reference 1. It is an object of the invention to provide further and improved ways of preparing vaccines that can raise immunity against both seasonal and pandemic strains.

### DISCLOSURE OF THE INVENTION

The invention provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1, characterized in that the concentration of each of the H1N1, H3N2 and B antigens in the first container is in the range 25-35 μg/ml e.g. about 30 μg/ml (typically at a 1:1:1 mass ratio). The concentration of the pandemic antigen in the second container is ideally in the range 5-20 μg/ml e.g. about 15 μg/ml. These antigen concentrations are, as is usual for inactivated influenza vaccines, expressed in terms of influenza virus hemagglutinin.

Keeping the pandemic vaccine separate from the seasonal vaccine, but within a kit, allows the strain profile of the final vaccine to be modified more easily than if a 4-valent vaccine is formulated during manufacture. This is particularly important due to strain fluctuation of pandemic viruses.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1, characterized in that the concentration of the pandemic antigen in the second container is in the range 5-20 μg/ml e.g. about 15 μg/ml.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1, characterized in that the volumes of aqueous vaccine in the first and second containers are substantially identical and are each in the range of about 0.4 ml to about 0.6 ml e.g. about 0.5 ml.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1, characterized in that the volume of the aqueous vaccine in the first container is substantially twice the volume of the aqueous vaccine in the second container.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1, characterized in that each of the containers is a borosilicate glass vial. Each vial may contain material for 1 dose or multiple (e.g. 10) doses. The vials may have stoppers made of butyl rubber.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the H5N1 influenza A virus strain is in clade 1, 2 or 4.

The invention also provides a kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, wherein the H5N1 strain is either selected from the group consisting of, or has a hemagglutinin that elicits anti-hemagglutinin antibodies that cross-react with hemagglutinin from a strain selected from the group consisting of A/Vietnam/1203/2004; A/Vietnam/1194/2004; A/duck/Hunan/795/2002; A/Indonesia/5/2005; A/Whooper-swan/Mongolia/244/2005; A/Chicken/India/NIV33487/2006; A/Anhui/1/2005; A/Bar-headed goose/Quighai/1A/2005; A/Japanese white eye/Hong Kong/1038/2006; A/turkey/Turkey/1/2005; A/goose/Guiyang/337/2006; A/duck/Laos/3295/2006; A/Cambodia/R0405050/2007; and A/common magpie/Hong Kong/5052/2007.

In some embodiments, the first container includes an adjuvant but the second container is unadjuvanted. In other embodiments, the first container includes an adjuvant and the second container also includes an adjuvant. In other embodiments, the first container is unadjuvanted but the second container includes an adjuvant. In other embodiments, the first and second containers are both unadjuvanted but the kit includes a third container that contains an oil-in-water emulsion adjuvant without antigen. In other embodiments, the first and second containers are both unadjuvanted but the kit includes a third container that contains an oil-in-water emulsion adjuvant and a non-influenza antigen. Details of suitable adjuvants are given below.

Using these kits the contents of the containers can be mixed at the time of use prior to convenient administration to a patient as a combined vaccine. The contents can be mixed in any suitable order. For instance, where the containers are vials, the contents of the second container can be withdrawn via a needle into a syringe and then inserted into the first container. After mixing, the mixed contents are withdrawn into a syringe (same as or different from before). The withdrawal needle may be used for injection, or may be discarded and replaced by one suitable for injection (e.g. IM injection).

The invention also provides a kit comprising: (i) a first container containing an aqueous inactivated influenza vaccine comprising antigen from a H1N1 influenza A virus strain, a H3N2 influenza A virus strain, a pandemic influenza A virus strain (e.g. a H5 strain, such as H5N1) and an influenza B virus strain; and (ii) a second container containing an oil-in-water emulsion adjuvant.

The invention provides a kit comprising: (i) a first container containing a lyophilized inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a pandemic strain e.g. a H5 strain, such as H5N1. The second container ideally includes an oil-in-water emulsion.

The invention provides a kit comprising: (i) a first container containing a lyophilized inactivated influenza vaccine comprising antigen from a H1N1 influenza A virus strain, a H3N2 influenza A virus strain, a pandemic influenza A virus strain (e.g. a H5 strain, such as H5N1) an influenza B virus strain; and (ii) a second container containing an oil-in-water emulsion adjuvant.

The invention also provides a syringe comprising multiple separate chambers, wherein: (i) a first chamber contains an aqueous inactivated influenza vaccine comprising antigen from a H1N1 influenza A virus strain, a H3N2 influenza A virus strain, and an influenza B virus strain; and (ii) a second chamber contains an aqueous inactivated influenza vaccine comprising antigen from a pandemic strain e.g. a H5 strain, such as H5N1. Thus the two aqueous components are held together but separately in the same syringe which, when actuated, results in mixing of the components. In some embodiments the first chamber includes an adjuvant. In other embodiments the second chamber includes an adjuvant. In other embodiments a third chamber includes an adjuvant. Multi-chamber syringes are known e.g. references 2-9 etc.

### Vaccine Preparation

Various forms of influenza virus vaccine are currently available, and vaccines are generally based either on live virus or on inactivated virus. Inactivated vaccines may be based on whole virions, split virions, or on purified surface antigens. Influenza antigens can also be presented in the form of virosomes. The invention can be used with any of these types of vaccine, but will typically be used with inactivated vaccines.

Where an inactivated virus is used, the vaccine may comprise whole virion, split virion, or purified surface antigens (including hemagglutinin and, usually, also including neuraminidase). Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Virions can be harvested from virus-containing fluids by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses are well known in the art e.g. see refs. 10-15, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. The BEGRIVAC™, FLUARIX™, FLUZONE™ and FLUSHIELD™ products are split vaccines.

Purified surface antigen vaccines comprise the influenza surface antigens haemagglutinin and, typically, also neuraminidase. Processes for preparing these proteins in purified form are well known in the art. The FLUVIRIN™, AGRIPPAL™ and INFLUVAC™ products are examples.

Another form of inactivated influenza antigen is the virosome [16] (nucleic acid free viral-like liposomal particles). Virosomes can be prepared by solubilization of influenza virus with a detergent followed by removal of the nucleocapsid and reconstitution of the membrane containing the viral glycoproteins. An alternative method for preparing virosomes involves adding viral membraneglycoproteins to excess amounts of phospholipids, to give liposomes with viral proteins in their membrane. The invention can be used to store bulk virosomes. as in the INFLEXAL V™ and INVAVAC™ products.

The influenza virus may be attenuated. The influenza virus may be temperature-sensitive. The influenza virus may be cold-adapted. These three features are particularly useful when using live virus as an antigen.

HA is the main immunogen in current inactivated influenza vaccines, and vaccine doses are standardised by reference to HA levels, typically measured by SRID. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses [17,18]). Except where otherwise stated, vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 1.9, about 1.5, etc. per strain. It is preferred to use substantially the same mass of HA for each strain included in the vaccine, except for the pandemic strain e.g. such that the HA mass for each strain is within 10% of the mean HA mass per strain, and preferably within 5% of the mean. The mass of HA for a pandemic strain is preferably ½ or ¼ the average mass for non-pandemic strains.

For live vaccines, dosing is measured by median tissue culture infectious dose (TCID₅₀) rather than HA content, and a TCID₅₀ of between 10⁶ and 10⁸ (preferably between 10^{6.5}-10^{7.5}) per strain is typical.

Strains used with the invention may have a natural HA as found in a wild-type virus, or a modified HA. For instance, it is known to modify HA to remove determinants (e.g. hyper-basic regions around the HA1/HA2 cleavage site) that cause a virus to be highly pathogenic in avian species. Hemagglutinins of influenza B viruses used with the invention preferably have Asn at amino acid 197, providing a glycosylation site [19].

### Cell Lines

Rather than use SPF eggs as the substrate for viral growth, where virus is harvested from infected allantoic fluids of hens' eggs, cell lines that support influenza virus replication may be used. The cell line will typically be of mammalian origin. Suitable mammalian cells of origin include, but are not limited to, hamster, cattle, primate (including humans and monkeys) and dog cells, although the use of primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line [20-22]. Suitable dog cells are e.g. kidney cells, as in the CLDK and MDCK cell lines.

Thus suitable cell lines include, but are not limited to: MDCK; CHO; CLDK; HKCC; 293T; BHK; Vero; MRC-5; PER.C6 [23]; FRhL2; WI-38; etc. Suitable cell lines are widely available e.g. from the American Type Cell Culture (ATCC) collection [24], from the Coriell Cell Repositories [25], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940.

The most preferred cell lines are those with mammalian-type glycosylation. As a less-preferred alternative to mammalian cell lines, virus can be grown on avian cell lines [e.g. refs. 26-28], including cell lines derived from ducks (e.g. duck retina) or hens. Examples of avian cell lines include avian embryonic stem cells [26,29] and duck retina cells [27]. Suitable avian embryonic stem cells, include the EBx cell line derived from chicken embryonic stem cells, EB45, EB14, and EB14-074 [30]. Chicken embryo fibroblasts (CEF) may also be used. Rather than using avian cells, however, the use of mammalian cells means that vaccines can be free from avian DNA and egg proteins (such as ovalbumin and ovomucoid), thereby reducing allergenicity.

The most preferred cell lines for growing influenza viruses are MDCK cell lines [31-34], derived from Madin Darby canine kidney. The original MDCK cell line is available from the ATCC as CCL-34, but derivatives of this cell line may also be used. For instance, reference 31 discloses a MDCK cell line that was adapted for growth in suspension culture ('MDCK 33016′, deposited as DSM ACC 2219). Similarly, reference 35 discloses a MDCK-derived cell line that grows in suspension in serum-free culture ('B-702′, deposited as FERM BP-7449). Reference 36 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 37 discloses MDCK cell lines with high susceptibility to infection, including ‘MDCK.5F1’ cells (ATCC CRL-12042). Any of these MDCK cell lines can be used.

Virus may be grown on cells in adherent culture or in suspension. Microcarrier cultures can also be used. In some embodiments, the cells may thus be adapted for growth in suspension.

Cell lines are preferably grown in serum-free culture media and/or protein free media. A medium is referred to as a serum-free medium in the context of the present invention in which there are no additives from serum of human or animal origin. The cells growing in such cultures naturally contain proteins themselves, but a protein-free medium is understood to mean one in which multiplication of the cells (e.g. prior to infection) occurs with exclusion of proteins, growth factors, other protein additives and non-serum proteins, but can optionally include proteins such as trypsin or other proteases that may be necessary for viral growth.

Cell lines supporting influenza virus replication are preferably grown below 37° C. [38] (e.g. 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture e.g. before inoculation, at the same time as inoculation, or after inoculation [38].

In preferred embodiments, particularly with MDCK cells, a cell line is not passaged from the master working cell bank beyond 40 population-doubling levels.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [39]. Absence of herpes simplex viruses is particularly preferred.

### Host Cell DNA

Where virus has been grown on a cell line then it is standard practice to minimize the amount of residual cell line DNA in the final vaccine, in order to minimize any oncogenic activity of the DNA.

Thus a vaccine composition prepared according to the invention preferably contains less than 10 ng (preferably less than 1 ng, and more preferably less than 100 pg) of residual host cell DNA per dose, although trace amounts of host cell DNA may be present.

Vaccines containing <10 ng (e.g. <1 ng, <100 pg) host cell DNA per 15 μg of haemagglutinin are preferred, as are vaccines containing <10 ng (e.g. <1 ng, <100 pg) host cell DNA per 0.25 ml volume. Vaccines containing <10 ng (e.g. <1 ng, <100 pg) host cell DNA per 50 μg of haemagglutinin are more preferred, as are vaccines containing <10 ng (e.g. <1 ng, <100 pg) host cell DNA per 0.5 ml volume.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 40 & 41, involving a two-step treatment, first using a DNase (e.g. Benzonase), which may be used during viral growth, and then a cationic detergent (e.g. CTAB), which may be used during virion disruption. Removal by β-propiolactone treatment can also be used.

Measurement of residual host cell DNA is now a routine regulatory requirement for biologicals and is within the normal capabilities of the skilled person. The assay used to measure DNA will typically be a validated assay [42,43]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g. against known standard quantities of host cell DNA) and tested then quantitative DNA measurements can be routinely performed. Three main techniques for DNA quantification can be used: hybridization methods, such as Southern blots or slot blots [44]; immunoassay methods, such as the Threshold™ System [45]; and quantitative PCR [46]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [45]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 47.

### Strain Selection

Vaccines produced according to the invention include at least four influenza virus strains. The different strains will typically be grown separately and then mixed after the viruses have been harvested and antigens have been prepared. Thus a process of the invention may include the step of mixing antigens from more than one influenza strain.

Influenza A virus currently displays sixteen HA subtypes: H1, H2, H3, H4, H5, H6, H7, H8, H9, H10, H11, H12, H13, H14, H15 and H16. The invention may protect against one or more of influenza A virus NA subtypes N1, N2, N3, N4, N5, N6, N7, N8 or N9. Vaccines herein include at least a H1 strain, a H3 strain and a pandemic strain. In some embodiments the H3 strain cross-reacts with A/Moscow/10/99. In other embodiments the H3 strain cross-reacts with A/Fujian/411/2002.

Characteristics of a pandemic influenza strain are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the vaccine recipient and the general human population are immunologically naïve to the strain's hemagglutinin; (b) it is capable of being transmitted horizontally in the human population; and (c) it is pathogenic to humans. Pandemic strains can include H2, H5, H7 or H9 subtype strains e.g. H5N1, H5N3, H9N2, H2N2, H7N1 and H7N7 strains. H5N1 strains are typical. Other pandemic strains for use with the invention may be derived from the H1N1 strains which transferred from swines to humans in 2009. Thus the pandemic strain may be a H1 strain with a hemagglutinin which is more closely related to SEQ ID NO: 14 than to SEQ ID NO: 15 (i.e. when aligned with the same algorithm and parameters, it has a higher degree sequence identity to SEQ ID NO: 14 than to SEQ ID NO: 15).

Influenza B virus currently does not display different HA subtypes, but influenza B virus strains do fall into two distinct lineages. These lineages emerged in the late 1980s and have HAs which can be antigenically and/or genetically distinguished from each other [48]. Current influenza B virus strains are either B/Victoria/2/87-like or B/Yamagata/16/88-like. These strains are usually distinguished antigenically, but differences in amino acid sequences have also been described for distinguishing the two lineages e.g. B/Yamagata/16/88-like strains often (but not always) have HA proteins with deletions at amino acid residue 164, numbered relative to the ‘Lee40’ HA sequence [49]. In some embodiments the influenza B strain is B/Victoria/2/87-like. In other embodiments the influenza B strain is B/Yamagata/16/88-like.

An influenza virus used with the invention may be a reassortant strain, and may have been obtained by reverse genetics techniques. Reverse genetics techniques [e.g. 50-54] allow influenza viruses with desired genome segments to be prepared in vitro using plasmids. Typically, it involves expressing (a) DNA molecules that encode desired viral RNA molecules e.g. from poll promoters or bacteriophage RNA polymerase promoters, and (b) DNA molecules that encode viral proteins e.g. from polII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA can be used [55-57], and these methods will also involve the use of plasmids to express all or some (e.g. just the PB1, PB2, PA and NP proteins) of the viral proteins, with up to 12 plasmids being used in some methods. To reduce the number of plasmids needed, a recent approach [58] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 58 method involve: (a) PB1, PB2 and PA mRNA-encoding regions on a single plasmid; and (b) all 8 vRNA-encoding segments on a single plasmid. Including the NA and HA segments on one plasmid and the six other segments on another plasmid can also facilitate matters.

As an alternative to using polI promoters to encode the viral RNA segments, it is possible to use bacteriophage polymerase promoters [59]. For instance, promoters for the SP6, T3 or T7 polymerases can conveniently be used. Because of the species-specificity of poll promoters, bacteriophage polymerase promoters can be more convenient for many cell types (e.g. MDCK), although a cell must also be transfected with a plasmid encoding the exogenous polymerase enzyme.

In other techniques it is possible to use dual polI and polII promoters to simultaneously code for the viral RNAs and for expressible mRNAs from a single template [60,61].

Thus an influenza A virus may include one or more RNA segments from a A/PR/8/34 virus (typically 6 segments from A/PR/8/34, with the HA and N segments being from a vaccine strain, i.e. a 6:2 reassortant). It may also include one or more RNA segments from a A/WSN/33 virus, or from any other virus strain useful for generating reassortant viruses for vaccine preparation. An influenza A virus may include fewer than 6 (i.e. 0, 1, 2, 3, 4 or 5) viral segments from an AA/6/60 influenza virus (A/Ann Arbor/6/60). An influenza B virus may include fewer than 6 (i.e. 0, 1, 2, 3, 4 or 5) viral segments from an AA/1/66 influenza virus (B/Ann Arbor/1/66). Typically, the invention protects against a strain that is capable of human-to-human transmission, and so the strain's genome will usually include at least one RNA segment that originated in a mammalian (e.g. in a human) influenza virus. It may include NS segment that originated in an avian influenza virus.

Strains whose antigens can be included in the compositions may be resistant to antiviral therapy (e.g. resistant to oseltamivir [62] and/or zanamivir), including resistant pandemic strains [63].

Particularly useful strains are those that have not been passaged through eggs at any stage between isolation from a patient and replication in a cell culture system, inclusive. MDCK cells can be used exclusively of for all steps from isolation to virus replication.

In some embodiments, strains used with the invention have hemagglutinin with a binding preference for oligosaccharides with a Sia(α2,6)Gal terminal disaccharide compared to oligosaccharides with a Sia(α2,3)Gal terminal disaccharide. Human influenza viruses bind to receptor oligosaccharides having a Sia(α2,6)Gal terminal disaccharide (sialic acid linked α-2,6 to galactose), but eggs and Vero cells have receptor oligosaccharides with a Sia(α2,3)Gal terminal disaccharide. Growth of human influenza viruses in cells such as MDCK provides selection pressure on hemagglutinin to maintain the native Sia(α2,6)Gal binding, unlike egg passaging.

To determine if a virus has a binding preference for oligosaccharides with a Sia(α2,6)Gal terminal disaccharide compared to oligosaccharides with a Sia(α2,3)Gal terminal disaccharide, various assays can be used. For instance, reference 64 describes a solid-phase enzyme-linked assay for influenza virus receptor-binding activity which gives sensitive and quantitative measurements of affinity constants. Reference 65 used a solid-phase assay in which binding of viruses to two different sialylglycoproteins was assessed (ovomucoid, with Sia(α2,3)Gal determinants; and pig α₂-macroglobulin, which Sia(α2,6)Gal determinants), and also describes an assay in which the binding of virus was assessed against two receptor analogs: free sialic acid (Neu5Ac) and 3′-sialyllactose (Neu5Acα2-3Galβ1-4Glc). Reference 66 reports an assay using a glycan array which was able to clearly differentiate receptor preferences for α2,3 or α2,6 linkages. Reference 67 reports an assay based on agglutination of human erythrocytes enzymatically modified to contain either Sia(α2,6)Gal or Sia(α2,3)Gal. Depending on the type of assay, it may be performed directly with the virus itself, or can be performed indirectly with hemagglutinin purified from the virus.

In some embodiments influenza strains used with the invention have glycoproteins (including hemagglutinin) with a different glycosylation pattern from egg-derived viruses. Thus the glycoproteins will include glycoforms that are not seen in chicken eggs.

Where a pandemic strain is used then, except where otherwise indicated, it will usually be from an influenza A virus in the H5 hemagglutinin subtype, such as a H5N1 strain. Within the H5 subtype the strain can be in clade 0, 1, 2, 3, 4, 5, 6, 7, 8 or 9.

The haemagglutinin (HA) sequences of the majority of H5N1 viruses circulating in avian species since 2003 separate into 2 distinct phylogenetic clades. Clade 1 viruses circulating in Cambodia, Thailand and VietNam were responsible for human infections in those countries during 2004 and 2005, and in Thailand during 2006. Clade 2 viruses have circulated in birds in China and Indonesia since 2003; they spread westwards during 2005 and 2006 to the Middle East, Europe and Africa. Since late 2005, clade 2 viruses have been principally responsible for human infections. Multiple subclades of clade 2 have been distinguished; three of these—subclades 1, 2 and 3—differ in geographical distribution and have so far been largely responsible for human cases. Further sub-division is also known e.g. within clade 2.3 there are four divisions including 2.3.2 and 2.3.4

Between August 2006 and March 2007, the majority of HA sequences of H5N1 viruses that have continued to circulate or have re-emerged in avian species and have been associated with sporadic human infections in Africa, Asia and Europe, fell into the previously designated phylogenetic clades and subclades. Clade 1 viruses were responsible for outbreaks in birds in Thailand and VietNam and for human infections in Thailand. Clade 2.1 viruses continue to circulate in poultry and cause human infections in Indonesia. Clade 2.2 viruses have caused outbreaks in birds in some countries in Africa, Asia and Europe, and have been associated with human infections in Egypt, Iraq and Nigeria. Clade 2.3 viruses have been isolated sporadically in Asia and have been responsible for human infections in China and the Lao People's Democratic Republic.

In addition, a few viruses that fall outside of these classifications were isolated from domestic poultry during localized outbreaks in Asia. These fall into emerging clades, represented by A/goose/Guiyang/337/2006 (clade 4) and A/chicken/Shanxi/2/2006 (clade 7). In total, 10 clades have currently been defined, numbered 0 to 9.

For reference herein, prototypic strains for each clade are as follows, together with the coding sequence of their hemagglutinin genes:

| Clade | Strain | SEQ ID NO |
|---|---|---|
| 1 | A/HongKong/213/03 | 1 |
| 2 | A/Indonesia/5/05 | 2 |
| 3 | A/Chicken/Hong Kong/SF219/01 | 3 |
| 4 | A/chicken/Guiyang/441/2006 | 4 |
| 5 | A/duck/Guangxi/1681/2004 | 5 |
| 6 | A/tree sparrow/Henan/4/2004 | 6 |
| 7 | A/chicken/Shanxi/2/2006 | 7 |
| 8 | A/Chicken/Henan/12/2004 | 8 |
| 9 | A/duck/Guangxi/2775/2005 | 9 |
| 0 | A/Hong Kong/156/97 | 10 |

A clade 1 H5 virus may be defined herein in phylogenetic terms as an influenza A virus having a hemagglutinin coding sequence that is more closely related to the coding sequence from the A/HongKong/213/03 strain (SEQ ID NO: 1) than to any the coding sequence from any of clades 0 and 2 to 9 (SEQ ID NOs: 2 to 10), when assessed using the DNADIST algorithm as implemented in the Phylip package [68] (e.g. using Kimura 2-parameter distances and a square matrix). Similarly, a clade 2 virus has a hemagglutinin coding sequence that is more closely related to the coding sequence from the A/Indonesia/5/05 strain (SEQ ID NO: 2) than to any the coding sequence from any of clades 0, 1 and 3 to 9 (SEQ ID NOs: 1 and 3 to 10). The other clades are phlyogenetically defined similarly—with a hemagglutinin coding sequence that is more closely related to the relevant coding sequence from SEQ ID NOs: 1 to 10 than to the other sequences in SEQ ID NOs: 1 to 10.

A clade 1 virus may be defined herein in nucleic acid sequence terms as an influenza A virus having a hemagglutinin coding sequence with greater sequence identity to the A/HongKong/213/03 strain (SEQ ID NO: 1) than to any of SEQ ID NOs: 2 to 10. The other clades are defined similarly—with a hemagglutinin coding sequence that is more closely related to the relevant coding sequence from SEQ ID NOs: 1 to 10 than to the other sequences in SEQ ID NOs: 1 to 10.

A H5 virus may be defined herein as being in a particular clade in amino acid sequence terms by reference to characteristic HA mutations [69]. For instance, a clade 3 virus may have one or more of the following amino acid residues, which are distinct from clades 1 and 2: Asn-45; Ser-84; Asn-94; Asn-124; Leu-138; Ser-144; Glu-212; Ser-223; and/or Arg-325. A clade 2 virus may have Asp-124, which is not seen in clades 1 and 3. A clade 1 virus may have one or more of the following amino acid residues, which are distinct from clades 2 and 3: Ser-124; Leu-129.

Within clade 2, at least three subclades have been recognized: 2.1, 2.2 and 2.3. A clade 2.1 H5 virus may be defined herein in phylogenetic terms as having a hemagglutinin coding sequence that is more closely related to the A/Indonesia/5/05 strain (SEQ ID NO: 2) than to either the A/Anhui/1/2005 strain (SEQ ID NO: 11) or the A/turkey/Turkey/1/05 (SEQ ID NO: 12). Similarly, a clade 2.2 H5 virus may be defined herein in phylogenetic terms as having a hemagglutinin coding sequence that is more closely related to the A/turkey/Turkey/1/05 strain (SEQ ID NO: 12) than to either the A/Anhui/1/2005 (SEQ ID NO: 11) or the A/Indonesia/5/05 strain (SEQ ID NO: 2). Finally, a clade 2.3 H5 virus may be defined herein in phylogenetic terms as having a hemagglutinin coding sequence that is more closely related to the A/Anhui/1/05 strain (SEQ ID NO: 11) than to either the A/turkey/Turkey/1/05 (SEQ ID NO: 12) or the A/Indonesia/5/05 strain (SEQ ID NO: 2).

In some embodiments a strain in subclade 2.2 may have HA including one or more of the following sequences: Ile-223; Ile-230; Ile-517; ΔSer-133; a cleavage site having sequence REGRRRKR (SEQ ID NO: 13). The HA gene may include one or more of nucleotides: A-41; A-142; A-209; A-295; G-433; A-467; A-496; C-610; A-627; A-643; C-658; T-661; T-689; T-727; G-880; C-937; G-1006; T-1012; A-1019; T-1177; A-1235; T-1402; C-1415; T-1480; C-1510; T-1614; C-1615; A-1672; G-1708 (any of which may or may not change the encoded amino acid for the relevant codon). The NA gene may include nucleotide A-743, which will not change the encoded amino acid for the relevant codon.

Usefully, in a 4-valent mixture the invention includes antigen from three different influenza A virus hemagglutinin subtypes (e.g. H1, H3, H5) but fewer than three different influenza A virus neuraminidase subtypes (e.g. two: N1 and N2). This arrangement comes from using multiple influenza A virus strains which share a common neuraminidase subtype e.g. H1N1 and H5N1. This common N subtype can enhance cross-protection [70].

### Pharmaceutical Compositions

Kits of the invention can be used for extemporaneous preparation of immunogenic pharmaceutical compositions (e.g. vaccines). Such compositions usually include components in addition to the influenza antigens e.g. they typically include one or more pharmaceutical carrier(s) and/or excipient(s). A thorough discussion of such components is available in reference 71. In many embodiments adjuvants may also be included.

Compositions will generally be in aqueous form at the point of administration.

A composition may include preservatives such as thiomersal or 2-phenoxyethanol. It is preferred that the vaccine should be substantially free from (e.g. <10 μg/ml) mercurial material e.g. thiomersal-free [14,72]. Vaccines containing no mercury are more preferred. Preservative-free vaccines are particularly preferred. α-tocopherol succinate can be included as an alternative to mercurial compounds [14]. Where antigen is present in more than one container in a kit, in some embodiments each of these containers includes a preservative, but in other embodiments only one antigen-containing container includes a preservative. Where adjuvant is in a separate container from antigens, the adjuvant is ideally mercury-free. After mixing, however, the origin of the preservative will usually not be apparent.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate, and/or magnesium chloride, etc. Where adjuvant is in a separate container from antigens, sodium chloride may be present in both containers.

Compositions may have an osmolality of between 200 mOsm/kg and 400 mOsm/kg, preferably between 240-360 mOsm/kg, maybe within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range. Where adjuvant is in a separate container from antigens, a buffer may be present in both containers.

The pH of a composition will generally be between 5.0 and 8.1, and more typically between 6.0 and 8.0 e.g. 6.5 and 7.5, or between 7.0 and 7.8. A process of the invention may therefore include a step of adjusting the pH of bulk vaccine prior to packaging.

The composition is preferably sterile. The composition is preferably non-pyrogenic e.g. containing <1 EU (endotoxin unit, a standard measure) per dose, and preferably <0.1 EU per dose. The composition is preferably gluten free.

Compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may included less than 1 mg/ml of each of octoxynol-10 and polysorbate 80. Other residual components in trace amounts could be antibiotics (e.g. neomycin, kanamycin, polymyxin B). Where adjuvant is in a separate container from antigens, this detergent will usually be present in the antigen-containing container (e.g. antigen with polysorbate 80 and Octoxynol 10).

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Influenza vaccines are typically administered in a dosage volume of about 0.5 ml, although a half dose (i.e. about 0.25 ml) may be administered to children. In some embodiments of the invention a composition may be administered in a higher dose e.g. about 1 ml e.g. after mixing two 0.5 ml volumes.

Compositions and kits are preferably stored at between 2° C. and 8° C. They should not be frozen. They should ideally be kept out of direct light.

### Adjuvants

At the point of use compositions of the invention may advantageously include an adjuvant, which can function to enhance the immune responses (humoral and/or cellular) elicited in a patient who receives the composition. The presence of an oil-in-water emulsion adjuvant (particularly one comprising squalene) has been shown to enhance the strain cross-reactivity of immune responses for seasonal [73] and pandemic [74,75] influenza vaccines.

An emulsion adjuvant may be included with a 3-valent seasonal kit component, a 1-valent pandemic kit component, or as a separate kit component.

Oil-in-water emulsions for use with the invention typically include at least one oil and at least one surfactant, with the oil(s) and surfactant(s) being biodegradable (metabolisable) and biocompatible. The oil droplets in the emulsion are generally less than 5 μm in diameter, and may even have a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil, etc. In the grain group, corn oil is the most readily available, but the oil of other cereal grains such as wheat, oats, rye, rice, teff, triticale, etc. may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, are readily available from commercial sources or may be obtained by methods known in the art. Squalene is preferred.

Other useful oils are the tocopherols, which are advantageously included in vaccines for use in elderly patients (e.g. aged 60 years or older) because vitamin E has been reported to have a positive effect on the immune response in this patient group [76]. They also have antioxidant properties that may help to stabilize the emulsions [77]. Various tocopherols exist (α, β, γ, δ, ε or ξ) but α is usually used. A preferred α-tocopherol is DL-α-tocopherol. α-tocopherol succinate is known to be compatible with influenza vaccines and to be a useful preservative as an alternative to mercurial compounds [14].

Mixtures of oils can be used e.g. squalene and α-tocopherol. An oil content in the range of 2-20% (by volume) is typical.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. The most preferred surfactant for including in the emulsion is polysorbate 80 (polyoxyethylene sorbitan monooleate; Tween 80).

Mixtures of surfactants can be used e.g. Tween 80/Span 85 mixtures. A combination of a polyoxyethylene sorbitan ester and an octoxynol is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Squalene-containing oil-in-water emulsions are preferred, particularly those containing polysorbate
80. Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [78-80], as described in more detail in Chapter 10 of ref. 81 and chapter 12 of ref. 82. The MF59 emulsion advantageously includes citrate ions e.g. 10 mM sodium citrate buffer.
A submicron emulsion of squalene, a tocopherol, and polysorbate 80. These emulsions may have from 2 to 10% squalene, from 2 to 10% tocopherol and from 0.3 to 3% polysorbate 80, and the weight ratio of squalene:tocopherol is preferably ≦1 (e.g. 0.90) as this can provide a more stable emulsion. Squalene and polysorbate 80 may be present at a volume ratio of about 5:2 or at a weight ratio of about 11:5. One such emulsion can be made by dissolving Tween 80 in PBS to give a 2% solution, then mixing 90 ml of this solution with a mixture of (5g of DL-α-tocopherol and 5 ml squalene), then microfluidising the mixture. The resulting emulsion has submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [83].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μml polysorbate 80, 110 μml Triton X-100 and 100 μ/ml α-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [84] (0.05-1% Thr-MDP, 5% squalane, 2.5% Pluronic L121 and 0.2% polysorbate 80). It can also be used without the Thr-MDP, as in the “AF” adjuvant [85] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [86]. The emulsion may also include one or more of: alditol; a cryoprotective agent (e.g. a sugar, such as dodecylmaltoside and/or sucrose); and/or an alkylpolyglycoside. The emulsion may include a TLR4 agonist [87]. Such emulsions may be lyophilized.
An emulsion of squalene, poloxamer 105 and Abil-Care [88]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 89, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 90, produced by addition of aliphatic amine to desacylsaponin via the carboxyl group of glucuronic acid), dimethyldioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [91].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [92].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [92].

The emulsions, whether in separate form or already mixed with at least one antigen, are mixed with a separate antigen-containing component extemporaneously, at the time of delivery. Where these two components are liquids then the volume ratio of the two liquids for mixing can vary (e.g. between 5:1 and 1:5) but is generally about 1:1.

After the antigen and adjuvant have been mixed, haemagglutinin antigen will generally remain in aqueous solution but may distribute itself around the oil/water interface. In general, little if any haemagglutinin will enter the oil phase of the emulsion.

### Packaging of Kit Components

Suitable containers for kit components of the invention include vials, syringes (e.g. disposable syringes), etc. These containers should be sterile. The containers can be packaged together to form a kit e.g. in the same box.

Where a component is located in a vial, the vial can be made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Useful vials are made of colorless glass. Borosilicate glasses are preferred to soda lime glasses. Vials may have stoppers made of butyl rubber.

A vial can have a cap (e.g. a Luer lock) adapted such that a syringe can be inserted into the cap. V vial cap may be located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. The plunger in a syringe may have a stopper to prevent the plunger from being accidentally removed during aspiration. The syringe may have a latex rubber cap and/or plunger. Disposable syringes contain a single dose of vaccine. The syringe will generally have a tip cap to seal the tip prior to attachment of a needle, and the tip cap may be made of a butyl rubber. If the syringe and needle are packaged separately then the needle is preferably fitted with a butyl rubber shield. Useful syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

A kit or composition may be packaged (e.g. in the same box) with a leaflet including details of the vaccine e.g. instructions for administration, details of the antigens within the vaccine, etc.

It is usual in multi-component products to include more material than is needed for patient administration, so that a full final dose volume is obtained despite any inefficiency in material transfer. Thus an individual container may include overfill e.g. of 5-20% by volume.

### Methods of Treatment, and Administration of the Vaccine

After mixing, compositions of the invention are suitable for administration to human patients, and the invention provides a method of raising an immune response in a patient, comprising the step of administering a mixed composition of the invention to the patient.

The invention also provides a method of raising an immune response in a patient, comprising the step of mixing the contents of the containers of a kit of the invention (or chambers of a syringe) and administering the mixed contents to the patient.

The invention also provides a kit of the invention for use as a medicament.

The invention also provides the use of A and B in the manufacture of a medicament for raising an immune response in a patient, where A is the contents of a first container and B is the contents of a second container, as defined above. The use may also include the use of C, wherein C is the contents of a third container, as defined above.

These methods and uses will generally be used to generate an antibody response, preferably a protective antibody response. Methods for assessing antibody responses, neutralising capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [93]. Antibody responses are typically measured by hemagglutination inhibition, by microneutralisation, by single radial immunodiffusion (SRID), and/or by single radial hemolysis (SRH). These assay techniques are well known in the art.

Compositions of the invention can be administered in various ways. The most preferred immunisation route is by intramuscular injection (e.g. into the arm or leg), but other available routes include subcutaneous injection, intranasal [94-96], oral [97], buccal, sublingual, intradermal [98,99], transcutaneous, transdermal [100], etc.

Vaccines prepared according to the invention may be used to treat both children and adults Influenza vaccines are currently recommended for use in pediatric and adult immunisation, from the age of 6 months. Thus the patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, immunodeficient patients, patients who have taken an antiviral compound (e.g. an oseltamivir or zanamivir compound; see below) in the 7 days prior to receiving the vaccine, people with egg allergies and people travelling abroad. The vaccines are not suitable solely for these groups, however, and may be used more generally in a population.

Preferred compositions of the invention satisfy 1, 2 or 3 of the CHMP criteria for efficacy. In adults (18-60 years), these criteria are: (1)≧70% seroprotection; (2)≧40% seroconversion; and/or (3) a GMT increase of ≧2.5-fold. In elderly (>60 years), these criteria are: (1)≧60% seroprotection; (2)≧30% seroconversion; and/or (3) a GMT increase of ≧2-fold. These criteria are based on open label studies with at least 50 patients.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients e.g. for people who have never received an influenza vaccine before, or for vaccines including a new HA subtype. Multiple doses will typically be administered at least 1 week apart (e.g. about 2 weeks, about 3 weeks, about 4 weeks, about 6 weeks, about 8 weeks, about 12 weeks, about 16 weeks, etc.).

In a typical immunization schedule, prior to receiving a composition of the invention (that includes at least 4 influenza strains, including A-H3, A-H1, A-H5 and B) a patient will have received at least one trivalent seasonal influenza vaccine (including A-H3, A-H1 and B antigens) and, separately, at least one pandemic influenza vaccine (typically a monovalent A-H5 vaccine). The 4-valent vaccine may be used to boost both of these immune responses. Thus the patient may already possess memory B cells that can differentiate into plasma cells that secrete antibodies against each of A-H3, A-H1, B and A-H5 hemagglutinins. Typically there will have been at least two prior doses of A-H5 vaccine.

The invention provides a method for raising an immune response in a patient against four strains of influenza virus (a H1N1 influenza A virus strain, a H3N2 influenza A virus strain, a pandemic influenza virus such as a H5N1 influenza A virus strain, and an influenza B virus strain), comprising steps of: (i) administering a first influenza virus vaccine, comprising antigen from a H1N1 influenza A virus, a H3N2 influenza A virus, and an influenza B virus; (ii) administering a second, influenza virus vaccine, comprising antigen from a pandemic influenza virus, such as H5N1 influenza A virus; and (iii) administering a third influenza virus vaccine, comprising antigen from a H1N1 influenza A virus, a H3N2 influenza A virus, the pandemic influenza virus and an influenza B virus, wherein steps (i) and (ii) may be performed either (a) simultaneously or (b) sequentially in either order, but before step (iii). The invention also provides a first vaccine and a second vaccine for use in this method. The first vaccine will typically be 3-valent, the second will be 1-valent, and the third will be 4-valent.

The invention also provides a method for raising (e.g. boosting) an immune response in a patient, comprising administering to a patient an influenza virus vaccine comprising antigen from a H1N1 influenza A virus, a H3N2 influenza A virus, a pandemic influenza virus (e.g. a H5 influenza A virus, such as H5N1) and an influenza B virus, wherein the patient has previously separately received both (a) an influenza virus vaccine (e.g. 3-valent) comprising antigen from a H1N1 influenza A virus, a H3N2 influenza A virus, and an influenza B virus and (b) an influenza virus vaccine (e.g. 1-valent) comprising antigen from the pandemic influenza virus.

The invention also provides a method for raising (e.g. boosting) an immune response in a patient, comprising administering to a patient an influenza virus vaccine comprising antigen from a H1N1 influenza A virus, a H3N2 influenza A virus, a pandemic influenza virus (e.g. a H5 influenza A virus, such as H5N1) and an influenza B virus, and then later administering separately (a) an influenza virus vaccine (e.g. 3-valent) comprising antigen from a H1N1 influenza A virus, a H3N2 influenza A virus, and an influenza B virus and (b) an influenza virus vaccine (e.g. 1-valent) comprising antigen from the pandemic influenza virus.

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C—W135-Y vaccine), a respiratory syncytial virus vaccine, a pneumococcal conjugate vaccine, etc. Administration at substantially the same time as a pneumococcal vaccine and/or a meningococcal vaccine is particularly useful in elderly patients.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3 (1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

### Specific Embodiments of First and Second Containers

In some instances the first container (or syringe chamber) includes a 3-valent influenza vaccine with antigens from H1N1 and H3N2 strains of influenza A virus and an influenza B virus and the second container (or syringe chamber) includes a 1-valent influenza vaccine with antigen from a H5N1 strain of influenza A virus e.g. of an A/VietNam/1194/2004 strain, such as NIBRG-14, or of an A/turkey/Turkey/1/05 strain, such as NIBRG-23.

In one embodiment, the 3-valent vaccine is a purified surface antigen vaccine prepared from viruses grown on eggs or MDCK cells, and the 1-valent vaccine is also a purified surface antigen vaccine prepared from virus grown on eggs or MDCK cells. The 3-valent vaccine is unadjuvanted, but the 1-valent vaccine is adjuvanted with a squalene-in-water emulsion. The 3-valent vaccine includes 15 μg HA per strain, but the 1-valent vaccine includes 7.5 μg HA. A human dose is made by mixing doses of the 3-valent and 1-valent vaccines at a 1:1 volume ratio e.g. to give a 1 ml dosing volume.

The 1-valent vaccine is made by mixing 0.25 ml of the emulsion (at 2× strength) with 0.25 ml of bulk antigen to give the desired final concentrations e.g. similar to FLUAD™ but with a ½ antigen dose of a H5N1 strain.

In another embodiment, the 3-valent vaccine is an inactivated vaccine (typically split) prepared from viruses grown on eggs, and the 1-valent vaccine is a split vaccine prepared from virus grown on eggs. The 3-valent vaccine is unadjuvanted, and the 1-valent vaccine is also adjuvanted, but they are supplied with a separate squalene-in-water emulsion. The 3-valent vaccine includes 15 μg HA per strain per dose, but the 1-valent vaccine includes 3.75 μg HA per dose. Thus the 4-valent mixed product includes, per dose, 15 μg HA for each of the H1N1, H3N2 and B viruses and 3.75 μg HA for the H5N1 virus. The antigen concentration in the 1-valent container can be 15 μg/ml. The 1-valent vaccine may include thimerosal (e.g. 20 μg/ml or 10 μg/ml). The emulsion comprises squalene, DL-α-tocopherol and polysorbate 80 at a weight ratio of 2.2:2.45:1 respectively e.g. 1068:1186:485. A human dose is made by mixing doses of the 3-valent and 1-valent vaccines and the emulsion e.g. 0.5 ml 3-valent, 0.25 ml 1-valent and 0.25 ml emulsion.

### Regimens

The invention also provides a regimen for administration of a 4-valent influenza vaccine, in which a patient receives two doses of 4-valent vaccine separated by at between 6 months and 18 months. The 4-valent vaccines may be prepared extemporaneously, as discussed above, or may be pre-mixed (e.g. distributed as 4-valent admixed vaccines by manufacturers).

Thus the invention provides a method for immunizing a human, comprising steps of (a) administering to the human a first 4-valent influenza vaccine; and then, between 180 and 540 days later, (b) administering to the same human a second 4-valent influenza vaccine.

The invention also provides first and second 4-valent influenza vaccines for use in immunizing a human by this method.

The invention also provides the use of first and second 4-valent influenza vaccines in the manufacture of medicaments for use in immunizing a human, wherein the first and second vaccines are administered to the same human between 180 and 540 days apart.

The invention also provides the use of a first 4-valent influenza vaccine in the manufacture of a medicament for pre-immunizing a human who will receive a second 4-valent influenza vaccine between 180 and 540 days later.

The invention also provides the use of a second 4-valent influenza vaccine in the manufacture of a medicament for immunizing a human, wherein the human had received a first 4-valent influenza vaccine between 180 and 540 days earlier than receiving the first vaccine.

The first 4-valent vaccine will typically include immunogens from: (i) a H1N1 influenza A virus strain; (ii) a H3N2 influenza A virus strain; (iii) a H5N1 influenza A virus strain; and (iv) an influenza B virus strain. The second 4-valent vaccine will also typically include immunogens from: (i) a H1N1 influenza A virus strain; (ii) a H3N2 influenza A virus strain; (iii) a H5N1 influenza A virus strain; and (iv) an influenza B virus strain. The first and second vaccines may be identical, but typically they will differ by at least one strain e.g. they may include hemagglutinins from different A/H1N1 strains and/or different A/H3N2 strains and/or different A/H5N1 strains and/or different B strains. Thus in some embodiments only one hemagglutinin is identical in the first 4-valent vaccine and the second 4-valent vaccine. In some embodiments only two hemagglutinins are identical in the first 4-valent vaccine and the second 4-valent vaccine. In some embodiments only three hemagglutinins are identical in the first 4-valent vaccine and the second 4-valent vaccine. In less typical embodiments all four hemagglutinins are identical in the first 4-valent vaccine and the second 4-valent vaccine.

Instead of including an immunogen from a H5N1 influenza A virus, the first and second vaccines may include an immunogen from another non-H1 and non-H3 influenza A subtype; for example from a H2, H5 (but not H5N1), H7 or H9 influenza A virus e.g. a H5N3, H9N2, H2N2, H7N1 or H7N7 strain.

The first and second 4-valent vaccines are usually of the same type i.e. both are live virus vaccines, both are whole virion vaccines, but are split virion vaccines, both are purified surface antigen vaccines, or both are virosome vaccines. Usually the first and second will both be inactivated.

The first and second 4-valent vaccines may both be adjuvanted. Suitable adjuvants are discussed above. In some embodiments, however, only the first vaccine is adjuvanted. In other less typical embodiments neither is adjuvanted.

The two vaccines are typically given in consecutive influenza seasons e.g. between 10-14 months apart, such as 11, 12 or 13 months apart.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, or for viral growth, it is preferably one that has been approved for use in human vaccine production e.g. as in Ph Eur general chapter 5.2.3.

### BRIEF DESCRIPTION OF DRAWINGS

There are no drawings

### MODES FOR CARRYING OUT THE INVENTION

A seasonal influenza vaccine was prepared including purified surface glycoproteins from A/H1N1, A/H3N2 and B strains. The antigen concentration was 30 μg HA per strain per ml, giving 15 μg/strain/dose.

A pre-pandemic vaccine was prepared based on purified surface glycoproteins from a H5N1 strain (clade 1) of influenza A virus. The final antigen concentration was 15 μg HA per ml, giving 7.5 μg/dose. The bulk antigen at 2× strength was mixed at a 1:1 volume ratio with MF59 emulsion adjuvant (2× strength) to give the final vaccine (1× strength).

0.5 ml of each of these two vaccines is mixed at the time of use and administered as a combination to patients, at a total volume of 1 ml. For comparison, other patients receive the two vaccines separately in different arms. Two control groups received either the seasonal or the pre-pandemic vaccine.

A total of 405 healthy adults (age 18-40 years) were randomized equally to 8 treatment groups (49-52 subjects/group). At day 1, three groups received the pre-pandemic vaccine in one arm and the seasonal vaccine concomitantly in the other arm (the ‘cone’ groups), and three other groups received an extemporaneous mixture of the pre-pandemic and seasonal vaccines (the ‘mix’ group). At day 22 the groups from the ‘cone’ and ‘mix’ groups received the pre-pandemic vaccine alone, the mixed vaccine, or no vaccine. Two crossover control groups received either the pre-pandemic or seasonal vaccine at day 1 but switched to receive the other vaccine at day 22.

Antibodies were evaluated by haemagglutination (HI), microneutralisation (MN) and single-radial haemolysis (SRH) on days 1, 22 and 43. Results were interpreted in the context of the CHMP criteria for assessment of seasonal vaccines (CPMP/BWP/214/96).

Baseline characteristics were generally similar between study groups, except for the proportion of subjects receiving influenza vaccination in the past (overall mean: 15%). In the HI analysis, all three CHMP criteria for vaccine immunologic efficacy were met after the first vaccination for all three seasonal strains in the 7 groups that received the seasonal vaccine. SRH assay results for the H5N1 strain satisfied all CHMP criteria after the second vaccination in the 4 study groups that received two doses of the pre-pandemic vaccine, regardless of the method used to co-administer the seasonal vaccine. Comparable results were observed for the MN H5N1 assay.

The adjuvanted H5N1 pre-pandemic vaccine is immunogenic and well tolerated when administered in combination with the seasonal influenza vaccine. The immune responses to seasonal influenza virus strains are similar whether the seasonal vaccine was administered alone, concomitantly with, or mixed with the pre-pandemic vaccine.

In separate work, 99 healthy adults 18-40 years of age received, as part of a larger study, one dose of pre-pandemic vaccine, either 3 weeks before or after one dose of a seasonal vaccine (2007 season). A booster H5N1 dose, using antigen from a different strain (clade 2) mixed with seasonal vaccine (2008 season) was given 1 year later. Antibodies against both H5 strains were measured one, two and three weeks later to assess crossreactivity by HI, MN and SRH, to assess seroconversion (SC) and seroprotection (≧40, SP).

After single-dose priming, the pre-pandemic vaccine gave similar SC (>40%) and SP (>40%) rates against homologous and heterologous viruses 3 weeks post-vaccination. Heterologous boosting produced anamnestic responses, evident within one week, indicative of immune memory in more than 90% of subjects, resulting in SC and SP rates equivalent to those seen after full two-dose homologous priming. The CHMP criteria for SC and SP were met and exceeded for both homologous and heterologous strains.

Thus a single shot of pre-pandemic vaccine is sufficient to prime the immune memory, which persists for at least one year. Furthermore, heterologous boosting results in anamnestic immune-response which exceeds CHMP criteria for licensing. Thus a heterologous strain of H5N1 can be used, in combination with a seasonal vaccine, as a booster one year after a single pre-pandemic priming dose.

It will be understood that the invention has been described by way of example only and modifications may be made whilst remaining within the scope and spirit of the invention.

### REFERENCES

### The Contents of which are Hereby Incorporated by Reference

[1] WO2008/068631.

[2] WO2005/089837.
[3] U.S. Pat. No. 6,692,468.
[4] WO00/07647.
[5] WO99/17820.
[6] U.S. Pat. No. 5,971,953.
[7] U.S. Pat. No. 4,060,082.
[8] EP-A-0520618.
[9] WO98/01174.
[10] WO02/28422.
[11] WO02/067983.
[12] WO02/074336.
[13] WO01/21151.
[14] WO02/097072.
[15] WO2005/113756.
[16] Huckriede et al. (2003) Methods Enzymol 373:74-91.
[17] Treanor et al. (1996) J Infect Dis 173:1467-70.
[18] Keitel et al. (1996) Clin Diagn Lab Immunol 3:507-10.
[19] Saito et al. (2004) J Med Virol 74(2):336-43.
[20] Kistner et al. (1998) Vaccine 16:960-8.
[21] Kistner et al., (1999) Dev Biol Stand 98:101-110.
[22] Bruhl et al. (2000) Vaccine 19:1149-58.
[23] Pau et al. (2001) Vaccine 19:2716-21.
[24] http://www.atcc.org/
[25] http://locus.umdnj.edu/
[26] WO03/076601.
[27] WO2005/042728.
[28] WO03/043415.
[29] WO01/85938.
[30] WO2006/108846.
[31] WO97/37000.
[32] Brands et al. (1999) Dev Biol Stand 98:93-100.
[33] Halperin et al. (2002) Vaccine 20:1240-7.
[34] Tree et al. (2001) Vaccine 19:3444-50.
[35] EP-A-1260581 (WO01/64846).
[36] WO2006/071563.
[37] WO2005/113758.
[38] WO97/37001.
[39] WO2006/027698.
[40] EP-B-0870508.
[41] U.S. Pat. No. 5,948,410.
[42] Lundblad (2001) Biotechnology and Applied Biochemistry 34:195-197.
[43] Guidance for Industry: Bioanalytical Method Validation. U.S. Department of Health and Human Services Food and Drug Administration Center for Drug Evaluation and Research (CDER) Center for Veterinary Medicine (CVM). May 2001.
[44] Ji et al. (2002) Biotechniques. 32:1162-7.
[45] Briggs (1991) J Parenter Sci Technol. 45:7-12.
[46] Lahijani et al. (1998) Hum Gene Ther. 9:1173-80.
[47] Lokteff et al. (2001) Biologicals. 29:123-32.
[48] Rota et al. (1992) J Gen Virol 73:2737-42.
[49] GenBank sequence GI:325176.
[50] Hoffmann et al. (2002) Vaccine 20:3165-3170.
[51] Subbarao et al. (2003) Virology 305:192-200.
[52] Liu et al. (2003) Virology 314:580-590.
[53] Ozaki et al. (2004) J. Virol. 78:1851-1857.
[54] Webby et al. (2004) Lancet 363:1099-1103.
[55] WO00/60050.
[56] WO01/04333.
[57] U.S. Pat. No. 6,649,372.
[58] Neumann et al. (2005) Proc Natl Acad Sci USA 102:16825-9.
[59] WO2006/067211.
[60] WO01/83794.
[61] Hoffmann et al. (2000) Virology 267(2):310-7.
[62] Herlocher et al. (2004) J Infect Dis 190(9):1627-30.
[63] Le et al. (2005) Nature 437(7062):1108.
[64] Gambaryan & Matrosovich (1992) J Virol Methods 39(1-2):111-23.
[65] Mastrosovich et al. (1999) J Virol 73: 1146-55.
[66] Stevens et al. (2006) J Mol Biol 355:1143-55.
[67] Couceiro & Baum (1994) Mem Inst Oswaldo Cruz 89(4):587-91.
[68] Felsenstein (1989) Cladistics 5: 164-166.
[69] Emerging Infectious Diseases 11(10):1515-21.
[70] Sandbulte et al. (2007) PLoS Med. 4(2):e59.
[71] Gennaro (2000) Remington: The Science and Practice of Pharmacy. 20th edition, ISBN: 0683306472.
[72] Banzhoff (2000) Immunology Letters 71:91-96.
[73] O'Hagan (2007) Expert Rev Vaccines. 6(5):699-710.
[74] Bernstein et al. (2008) J Infect Dis. 197(5):667-75.
[75] Stephenson et al. (2005) J Infect Dis. 191(8):1210-5.
[76] Han et al. (2005) Impact of Vitamin E on Immune Function and Infectious Diseases in the Aged at Nutrition, Immune functions and Health EuroConference, Paris, 9-10 Jun. 2005.
[77] U.S. Pat. No. 6,630,161.
[78] WO90/14837.
[79] Podda & Del Giudice (2003) Expert Rev Vaccines 2:197-203.
[80] Podda (2001) Vaccine 19: 2673-2680.
[81] Vaccine Design: The Subunit and Adjuvant Approach (eds. Powell & Newman) Plenum Press 1995 (ISBN 0-306-44867-X).
[82] Vaccine Adjuvants: Preparation Methods and Research Protocols (Volume 42 of Methods in Molecular Medicine series). ISBN: 1-59259-083-7. Ed. O'Hagan.
[83] WO2008/043774.
[84] Allison & Byars (1992) Res Immunol 143:519-25.
[85] Hariharan et al. (1995) Cancer Res 55:3486-9.
[86] US-2007/0014805.
[87] US-2007/0191314.
[88] Suli et al. (2004) Vaccine 22(25-26):3464-9.
[89] WO95/11700.
[90] U.S. Pat. No. 6,080,725.
[91] WO2005/097181.
[92] WO2006/113373.
[93] Potter & Oxford (1979) Br Med Bull 35: 69-75.
[94] Greenbaum et al. (2004) Vaccine 22:2566-77.
[95] Zurbriggen et al. (2003) Expert Rev Vaccines 2:295-304.
[96] Piascik (2003) J Am Pharm Assoc (Wash D.C.). 43:728-30.
[97] Mann et al. (2004) Vaccine 22:2425-9.
[98] Halperin et al. (1979) Am J Public Health 69:1247-50.
[99] Herbert et al. (1979) J Infect Dis 140:234-8.
Chen et al. (2003) Vaccine 21:2830-6.

1. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the concentration of each of the H1N1, H3N2 and B antigens in the first container is in the range 25-35 μg/ml (hemagglutinin).

2. The kit of claim 1, wherein the concentration of the H5N1 antigen in the second container is in the range 5-20 μg/ml (hemagglutinin) e.g. in the range 10-20 μg/ml (hemagglutinin).

3. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the concentration of H5N1 hemagglutinin antigen in the second container is from 5-20 μm/ml.

4. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the volumes of aqueous vaccine in the first and second containers are substantially identical and are each in the range of about 0.4 ml to about 0.6 ml.

5. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the volume of the aqueous vaccine in the first container is substantially twice the volume of the aqueous vaccine in the second container.

6. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that each of the containers is a borosilicate glass vial.

7. A kit comprising: (i) a first container containing an aqueous inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain, characterized in that the H5N1 influenza A virus strain is in clade 1, 2 or 4.

8. The kit of claim 7, wherein the H5N1 influenza A virus strain is in clade 2.1, 2.2 or 2.3.

9. The kit of any one of claims 1 to 8, wherein the first container is unadjuvanted but the second container includes an adjuvant.

10. The kit of any one of claims 1 to 8, wherein first and second containers are both unadjuvanted and the kit includes a third container that contains an oil-in-water emulsion adjuvant.

11. A kit comprising: (i) a first container containing an aqueous inactivated influenza vaccine comprising antigen from a H1N1 influenza A virus strain, a H3N2 influenza A virus strain, a H5N1 influenza A virus strain and an influenza B virus strain; and (ii) a second container containing an oil-in-water emulsion adjuvant.

12. A kit comprising: (i) a first container containing a lyophilized inactivated influenza vaccine comprising antigen from a H1N1 influenza A virus strain, a H3N2 influenza A virus strain, and an influenza B virus strain; and (ii) a second container containing an aqueous inactivated influenza vaccine comprising antigen from a H5N1 influenza A virus strain.

13. The kit of claim 12, wherein the second container includes an oil-in-water emulsion.

14. A kit comprising: (i) a first container containing a lyophilized inactivated influenza vaccine comprising antigen from a H1N1 influenza A virus strain, a H3N2 influenza A virus strain, a H5N1 influenza A virus strain an influenza B virus strain; and (ii) a second container containing an oil-in-water emulsion adjuvant.

15. A syringe comprising multiple separate chambers, wherein: (i) a first chamber contains an aqueous inactivated influenza vaccine comprising antigen from a H1N1 influenza A virus strain, a H3N2 influenza A virus strain, and an influenza B virus strain; and (ii) a second chamber contains an aqueous inactivated influenza vaccine comprising antigen from a H5N1 influenza A virus strain.

16. The syringe of claim 15, wherein the H1N1 influenza A virus strain, the H3N2 influenza A virus strain, the influenza B virus strain and the H5N1 influenza A virus strain are all grown in eggs.

17. The syringe of claim 15, wherein the H1N1 influenza A virus strain, the H3N2 influenza A virus strain, the influenza B virus strain and the H5N1 influenza A virus strain are all grown in cell culture.

18. The syringe of claim 17, wherein the containers are free from avian DNA, ovalbumin and ovomucoid.

19. The syringe of claim 15, wherein the H1N1 influenza A virus strain, the H3N2 influenza A virus strain and the influenza B virus strain are grown in a first substrate and the H5N1 influenza A virus strain is grown in a second substrate, wherein one of the substrates is egg and the other substrate is cell culture.

20. A method for raising an immune response in a patient against four strains of influenza virus (a H1N1 influenza A virus strain, a H3N2 influenza A virus strain, a H5N1 influenza A virus strain, and an influenza B virus strain), comprising steps of: (i) administering a first influenza virus vaccine, comprising antigen from a H1N1 influenza A virus, a H3N2 influenza A virus, and an influenza B virus; (ii) administering a second, influenza virus vaccine, comprising antigen from a H5N1 influenza A virus, such as H5N1 influenza A virus; and (iii) administering a third influenza virus vaccine, comprising antigen from a H1N1 influenza A virus, a H3N2 influenza A virus, a H5N1 influenza A virus and an influenza B virus, wherein steps (i) and (ii) may be performed either (a) simultaneously or (b) sequentially in either order, but before step (iii).

21. A method for raising an immune response in a patient, comprising administering to a patient an influenza virus vaccine comprising antigen from a H1N1 influenza A virus, a H3N2 influenza A virus, a H5N1 influenza A virus and an influenza B virus, wherein the patient has previously separately received both (a) a 3-valent influenza virus vaccine comprising antigen from a H1N1 influenza A virus, a H3N2 influenza A virus, and an influenza B virus and (b) a monovalent influenza virus vaccine comprising antigen from a H5N1 influenza A virus.

22. A method for raising an immune response in a patient, comprising administering to a patient an influenza virus vaccine comprising antigen from a H1N1 influenza A virus, a H3N2 influenza A virus, a H5N1 influenza A virus and an influenza B virus, and then later administering separately (a) a 3-valent influenza virus vaccine comprising antigen from a H1N1 influenza A virus, a H3N2 influenza A virus, and an influenza B virus and (b) a monovalent influenza virus vaccine comprising antigen from a H5N1 influenza A virus.


SEQUENCE LISTING (15 sequences)

SEQ ID NO: 1 - DNA, 1707 bp, Influenza A virus (A/Hong Kong/213/03)
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaacgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagccaatgacctctgttacccaggggatttcaacgactatgaagaattgaaacacctattgagcagaataaaccattttgagaaaattcagatcatccccaaaaattcttggtccagtcatgaagcctcattaggggtgagctcagcatgtccataccaaggaaagtcctcctttttcaggaatgtggtatggcttatcaaaaagaacaatgcatacccaacaataaagaggagctacaataataccaaccaagaagatcttttggtattgtgggggattcaccatcctaatgatgcggcagagcagactaggctctatcaaaacccaaccacctacatttccgttgggacatcaacactaaaccagagattggtaccaaaaatagctactagatccaaagtaaacgggcaaaatggaaggatggagttcttctggacaattttaaaaccgaatgatgcaatcaacttcgagagcaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaataacttagaaaggagaatagagaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcacaaatgtgataatgaatgtatggaaagtgtaagaaacggaacgtatgactacccgcagtattcagaagaagcaagactaaaaagagaggaaataagtggagtaaaattggagtcaataggaacttaccaaatactgtcaatttattctacagtggcgagttccctagcactggcaatcatggtagctggtctatctttatggatgtgctccaatgggtcgttacaatgcagaatttgcatttaa

SEQ ID NO: 2 - DNA, 1707 bp, Influenza A virus (A/Indonesia/5/05)
atggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagtgagctcagcatgtccatacctgggaagtccctccttttttagaaatgtggtatggcttatcaaaaagaacagtacatacccaacaataaagaaaagctacaataataccaaccaagaagatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 3 - DNA, 1654 bp, Influenza A virus (A/Chicken/Hong Kong/SF219/01)
agtcttgttaaaagtgatcagatttgcattggttaccatgcaaacaactcgacagagcaggttgacacaataatggaaaagaacgttactgttacacatgcccaagacatattggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccagtccagccaatgacctctgttacccaggggatttcaacgactatgaagaactgaaacacctattgagcagaataaaccattttgagaaaattcagatcatccccaaaagttcttggtccaatcatgaagcctcatcaggggtgagctcagcatgtccataccttgggaagtcctcctttttcagaaatgtggtatggcttatcaaaaagaacagtacatacccaacaataaagaggagctataataataccaaccaagaagatcttttggtactgtgggggattcaccatcctaatgatgcggcagagcagacaaagctctatcaaaacccaaccacctatatttccgttggaacatcaacactaaaccagagattggtaccaaaaatagctactagatccaaagtaaacgggcaaagtggaagaatggagttcttctggacaattttaaagccgaatgatgctatcaatttcgagagtaatggaaatttcattgctccagaatatgcatacaaaattgtcaagaaaggggactcatcaattatgaaaagtgaattggaatatggtaactgcaacaccaagtggcaaactccaatgggggcgataaactctagtatgccattccacaacatacaccctctcaccatcggggaatgccccaaatatgtgaaatcaaacagattagtccttgcgactggactcagaaatacccctcaaagagagagaagaagaaaaaagagaggactatttggagctatagcaggttttatagagggaggatggcagggaatggtagatggttggtatgggtaccaccatagcaatgagcaggggagtggatacgctgcagacaaagaatccactcaaaaggcaatagatggagttaccaataaggtcaactcgatcattgacaaaatgaacactcagtttgaggccgttggaagggaatttaataacttagaaaggagaatagaaaatttaaacaagaagatggaagacggattcctagatgtctggacttataatgctgaacttctggttctcatggaaaatgagagaactctagactttcacgactcaaatgtcaagaacctttacgacaaggtccgactacagcttagggataatgcaaaggagctgggtaacggctgtttcgagttctatcacaaatgtgataatgaatgtatggaaagtgtaaaaaacggaacgtatgactacccgcagtattcagaagaagcaagactaaacagagaggaaataagtggagtaaaattggaatcaatgggaacttaccaaatactgtcaatttattcaacagtggcgagttccctagcactggcaatcatggtagctggtctatctttatggatgtgctccaatggatcgttac

SEQ ID NO: 4 - DNA, 1668 bp, Influenza A virus (A/chicken/Guiyang/441/2006)
atggagaaaatagtgcttcttcttgcaataatcagtcttgttaaaagtgatcagatttgcattggttaccatgcaaacaactcgacagtgcaggttgacacgataatggaaaagaatgttactgtaacacatgcccaagacatactggaaaagacacacaatgggaagctctgcagtctagatggagtgaagcctctaattttaagagattgtagtgtagctggatggctcctcggaaacccaatgtgtgacgaattcatcaatgtgcccgaatggtcttacatagtggagaaggccagtccagccaatgacctctgttacccaggggatttcaacgactacgaagaactgaaacacctattgagcagaataaaccattttgagaaaattcagatcatccccaaaagttcttggcccaatcatgaagcctcactaggggtgagctcagcatgtccatacctgggggagtcctcctttttcagaaatgtggtatggcttatcaaaaagaacagttcatacccaacaataaagaggagctacaataataccaaccaagaagatcttttagtattgtgggggatccatcaccctaatgatgcggcagagcagataaagctttatcaaaacccaaacacctatatttccgttggaacatcaacactaaaccagaggttggtaccaacaatagctactagatccaaagtaaacgggcaaagtggaaggatggagttcttctggacaattttaaagccgaatgatactatcaatttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaactggactcagaaatacccttcaaagagagagaaggagaaaaaagagaggactatttggagccatagcaggttttattgagggaggatggcagggaatggtagacggttggtatgggtaccaccatagcaatgagcaggggagtggatacgctgcagacaaagaatccactcaaaaggcaatagatggaatcaccaataaggtcaactcgatcattaacaaaatgaacactcagtttgaggccgttggacgggaatttagtaacttagaaaggagaatagaaaatttaaacaagaagatggaagacggattcctagatgtctggacttataatgctgaacttctggttctcatggaaaatgaaagaactctagactttcatgactcaaatgtcaagaacctttacgacaaagtccgactacagcttagggataatgcaaaagagctgggtaacggttgtttcgagttctatcacaaatgtgatgatgaatgtatggaaagtgtaaaaaacggaacgtatgactacccgcagtattcagaagaagctagactaaacagagaggagataaatggagtaaaattggaatcaatgggaacctaccaaatactgtcaatttactcaacagtggcgagttccctagcactggcaatcatggtagctggtctatctttatggatg

SEQ ID NO: 5 - DNA, 1698 bp, Influenza A virus (A/duck/Guangxi/1681/2004)
atggagaaaatagtgcttcttcttgcaatagtcagtcttgttaaaagtgatcagatttgcattggttaccatgcaaacaactcgacagagcaggttgatacaataatggaaaagaatgttactgttacacatgcccaagacatactggaaaagacacacaacgggaaactctgcgatctagatggagtgaagcctctaattttgagagattgtagtgttgcgggatggctcctcggaaacccaatgtgtgatgaattcatcaatgtgccggaatggtcttacatagtggagaaggccagtccagccaatgacctctgttacccaggagatttcaacgactatgaagaactaaaacacctattgagcagaataaatcattttgagaaaattcagatcatccccaaaagttcttggtccaatcatgaagcctcatcaggggtgagctcagcatgtccataccaggggaggccctcctttttcaggaatgtggtatggcttatcaaaaagaacagtgcataccctacaataaagaggagctacaataataccagtcaagaagatcttttggtactgtgggggattcaccatcctaatgatgcggcagagcagacaaagctctatcaaaacccaactacctatatttccgttggaacatcaacactaaaccagagattggtaccaaaaatagctactagatccaaagtaaacgggcagagtggaagaatggagttcttctggacaattttaaagccgaatgatgctatcaacttcgagagtaatggaaatttcattgctccagaatatgcctacaaaattgtcaagaaaggggactcagcaattatgaaaagtgaattggaatatggtaactgcaacaccaaatgtcaaactccaatgggggcgataaactctagtatgccattccacaacatacaccctctcaccatcggggaatgccccaagtatgtgaaatcaagcagattagtccttgcgacagggctcaggaatagccctcaaagagagataagaagaaaaaagagaggactatttggagctatagcaggctttatagagggaggatggcagggaatggtagatggttggtatgggtaccaccatagcaacgagcaggggagtggatacgctgcagacaaagaatccactcaaaaggcaatagatggagtcaccaataaagtcaattcgatcattgacaaaatgaacactcaatttgaggccgttggaagggaatttaataatttagaaaggagaatagagaatttaaacaaaaagatggaggacggattcctagatgtctggacttataatgctgaacttctggttctcatggaaaatgagagaactctagactttcatgactcaaatgtcaagaacctttacgacagggtccgactacagcttagggataatgcaaaggagctggggaacggttgtttcgagttctatcacaagtgtgacaatgaatgcatggaaagtgtaagaaacggaacgtatgactacccgcagtattcagaagaagcaagactaaagagagaggaaataagtggagtaaaattggaatcgataggaacttaccaaatattgtcaatttattcaacagtggcgagttccctagcactggcaatcatggtagctggtctatctttatggatgtgctccaatggatcgttacaatgcagaatt

SEQ ID NO: 6 - DNA, 1707 bp, Influenza A virus (A/tree sparrow/Henan/4/2004)
atggagaaaatagtgcttcttcttgcaatagtcagtcttgttaaaagtgatcagatttgcattggttaccatgcaaacaactcgacagagcaggttgacacaataatgcaaaagaacgttactgttacacatgcccaagacatactggaaaagacacacaacgggaagctctgcgatctagatggagtgaaacctctaattttaagagattgtagtgtagctggatggctcctcggaaacccaatgtgtgacgaattcatcaatgtgccggaatggtcttacatagtggagaaggccagtccagccaatgacctctgttacccaggggatttcaacgactatgaagaactgaaacacctattgagcagaataaaccattttgagaaaattcagatcatccccaaaagttcttggtccgatcatgaagcctcatcaggggtgagctcagcatgtccataccaggggagtccctcctttttcagaaatgtggtatggcttatcaaaaagaacagtgcatacccaacaataaagaggagctacaataataccaaccaagaagatcttttggtactgtgggggattcaccatcctaatgatgcggcagagcagacaaagctctatcaaaacccaaccacctatatttccgttggaacatcaacactaaaccagagattggtaccaaaaatagctactagatccaaagtaaatgggcaaagtggaagaatggagttcttctggacaattttaaagccgaatgacgctatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcgacagggctcagaaatagccctcaaagagagagaagaagaaaaaagagaggactatttggagctatagcaggttttatagagggaggatggcagggaatggtagatggttggtatgggtaccaccatagcaatgagcaggggagtggatacgctgcagacaaagaatccactcaaaaggcaatagatggagtcaccaataaggtcaactcgatcattgacaaaatgaacactcagtttgaggccgttggaagggaatttaataacttagaaaggagaatagaaaatttaaacaagaagatggaggacggattcctagatgtctggacttataatgctgaacttctggttctcatggaaaatgagagaactctagactttcatgactcaaatgtcaagaacctttacgaaaaggtccgactacaacttagggataatgcaaaggagctgggtaacggttgtttcgagttctatcacaaatgtgataatgaatgtatggaaagtgtaaaaaacggaacgtatgactacccgcagtattcagaagaagcaagactaaacagagaggaaataagtggagtaaaattggaatcaataggaacttaccaaatactgtcaatttattcaacagtggtgagttccctagcactggcaatcatggtagctggtctatctttatggatgtgctccaatggatcgttacaatgcagaatttgcatttaa

SEQ ID NO: 7 - DNA, 1707 bp, Influenza A virus (A/chicken/Shanxi/2/2006)
atggagaaaatagtgcttcttcttgcaataatcggtcttgttaaaagtgatcagatttgcgttggttaccatgcaaacaactcaacagagcaggttgacacaataatggaaaagaacgttactgttacacatgctcaagacatactggagaagacacacaacgggaagctctgcgacctagatggagtgaagcctctcattttgaaagattgtagtgtagctggatggctcctcggaaaccctatgtgtgacgaatttatcaatgtgtcggaatggtcttacatagtggagaaggccagtccagccaatggcctctgttacccaggggatttcaatgactatgaagaactgaaacacctattgagcagaataaaccattttgagaaaattaagatcatccccaaaagttcttggtccaatcatgaagcctcatcaggggtgagctcagcatgttcctatctggggaagccctcctttttcagaaatgtggtatggcttatcaaaaagaataatacatacccaccaataaaggtgaactacaccaataccaaccaagaagatcttttggtactgtgggggattcaccatcccaacgatgagacagagcagataaagatctatcaaaacccaaccacctatatttccgttggaacatcaacactaaaccagagattggtaccaaaaatagctactaaatcccaagtgaacgggcaaagtggaagaatggagttcttctggacaattttaaagccgaatgatgctatcaatttcgatagtaatggaaatttcattgctccagaatatgcatacaaaattgtcaagaaaggggactcagcgattatgaaaagtgaattggaatatggtaactgcaacaccaagtgtcaaactccaatgggggcgataaattctagtatgccattccacaacatacaccctctcaccatcggggaatgccccaaatatgtgaaatcaaacagattagtcctcgcgactggactcagaaatgcccctcaaagagagggaagaagaaaaaagagaggactatttggagccatagcagggtttatagagggaggatggcagggaatggtagatggttggtatgggtaccaccatagcaatgagcaggggagtggatactctgcagacaaagaatccactcaaaaggcaatagatggagtcaccaataaggtcaactcgatcattgacaaaatgaacactcagtttgagggcgttgttagggaaattaataacttagaaaggagaatagagaatttaaacaaaaagatggaagacggattcctagatgtctggacttataacgctgaacttctggttctcatggaaaatgagagaactctagactttcatgactcaaatgtcaagaacctttacgacaaggtccgactgcagcttagggataatgcaaaggagctgggtaacggttgtttcgaattctatcacaaatgtgataatgaatgtatggaaagtgtaaaaaacggaacgtatgactacccgcagtattcagaagaagcaagactaaacagagaggaaataagtggagtaaaattggaatcaatggtaacttaccaaatactgtcaatttattcaacagtggcgagttccctagcattggcaatcatggtggctggtctatctttatggatgtgctccaatggatcgttacaatgcagaatttgcatttga

SEQ ID NO: 8 - DNA, 1707 bp, Influenza A virus (A/Chicken/Henan/12/2004)
atggagaaaatagtgcttcttcttgcaatagtcagtcttgttaaaagtgatcagatttgcattggttaccatgcaaacaactcgacagagcaggttgacacaataatggaaaagaacgttactgttacacatgcccaagacatactggaaaagacacacaacgggaagctctgcgatctagatggagtgaagcctctaattttgagagattgtagtgtagctggatggctcctcggaaacccaatgtgtgacgaattcatcaatgtgccggaatggtcttacatagtggagaaggccagtccagccaatggcctctgttacccaggggatttcaacgactatgaagaactgaaacacctattgagcagaataaaccattttgagaaaattcagatcatccccaaaagttcttggtccaatcatgaagcctcatcaggggtgagctcagcatgcccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaggctctatcaaaacccaaccacctatatttccgttggaacatcaacactaaaccagagattggtaccaaaaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcaataaactctagtatgccattccacaacatacaccctctcaccatcggggaatgccccaaatatgtgaaatcaaacagattagtccttgcgactgggctcagaaatagccctcaaagagagagaagaagaaaaaagagaggactatttggagctatagcaggttttatagagggaggatggcagggaatggtagatggttggtatgggtaccaccatagcaatgagcaggggagtggatacgctgcagacaaagaatccactcaaaaggcaatagatggagtcaccaataaggtcaactcgatcattgacaaaatgaacactcagtttgaggccgttggaagggaatttaataacttagaaaggagaatagagaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcacaaatgtgataatgaatgtatggaaagtgtaagaaacggaacgtatgactacccgcagtattcagaagaagcaagactaaaaagagaggaaataagtggagtaaaattggaatcaataggaacttaccaaatactgtcaatttattcaacagtggcgagttccctagcactggcaatcatggtagctggtctatctttatggatgtgctccaatggatcgttacaatgcagaatttgcatttaa

SEQ ID NO: 9 - DNA, 1695 bp, Influenza A virus (A/duck/Guangxi/2775/2005)
atggagaaaatagtgcttcttcttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgacctagatggggtgaagcctctaattttgagagattgtagtgtagctggatggctcctcgggaacccaatgtgtgacgaattcatcaatgtaccggaatggtcttacatagtggagaaggccagtccagccaatgacctctgttacccaggtgatttcaacgattatgaagaactgaaacacctattgagcagaataaaccattttgagaaaattcagatcatccccaaaagttcttggcccaaccatgaagcctcatcaggggtgagctcagcatgtccatacctgggaaagccctcctttttcagaaatgtggtatggcttatcaaaaagaacagtgcatacccaacaataaagaggagctacaataataccaaccaagaagatcttttggtactgtgggggattcaccatcctaatgatgagacagagcagacaaagctctatcaaaacccaaccacttatatttccgttggaacatcaacactaaaccagagattggtaccaaaaatagctaccagatccaaagtaaacgggcaaagtggaaggatggagttcttctggacaattttaaaaccgaatgatgcaatcaacttcgagagtaatggaaatttcattgctccagaatatgcctacaaaattgtcaagaaaggggactcagcaattatgaaaagtgaattggaatatggtaactgcaacaccaaatgtcaaactccaatgggggcgataaactctagtatgccattccacaacatacaccctctcaccatcggggaatgccccaaatatgtgaaatcaaacagattagtccttgcgactgggctcagaaatagccctcaaagggagagaagaagaaaaaagagaggactatttggagctatagcaggttttatagagggaggatggcagggaatggtagatggttggtacggataccaccatagcaatgagcaggggagtggatacgctgcagacaaagaatccactcaaaaggcaatagatggagtcaccaataaggtcaactcgatcattgacaaaatgaacactcagtttgaggccgttggaagggaatttaataacttagaaaggagaatagagaatttaaacaagaagatggaagacgggttcctagatgtctggacttataatgctgaacttctggttctcatggaaaatgagcgaactctagactttcatgactcaaatgtcaagaacctttacgacaaggtccgactacagcttagggataatgcaaaagagctgggtaacggttgtttcgagttctatcacaaatgtgataatgaatgcatggaaagtgtaagaaacggaacgtatgactacccgcattattcagaagaagcaaggctaaaaagagaggaaataagtggagtaaaattggaatcaataggaacttaccaaatactgtcaatttattcaacagtggcaagttccctagcgctggcaatcatggtagctggtctatctttatggatgtgctccaatggttcgttacaatgcaga

SEQ ID NO: 10 - DNA, 1707 bp, Influenza A virus (A/Hong Kong/156/97)
atggaaagaacagtgcttcttcttgcaacagtcagtcttgttaaaagtgatcagatttgcattggttaccatgcaaacaactcgacagagcaggttgacacaataatggaaaagaatgttactgttacacatgcccaagacatactggaaaggacacacaacgggaagctctgcgatctaaatggagtgaagcctctcattttgagggattgtagtgtagctggatggctcctcggaaaccctatgtgtgacgaattcatcaatgtgccggaatggtcttacatagtggagaaggccagtccagccaatgacctctgttatccagggaatttcaacgactatgaagaactgaaacacctattgagcagaataaaccattttgagaaaattcagatcatccccaaaagttcttggtccaatcatgatgcctcatcaggggtgagctcagcatgtccataccttgggaggtcctcctttttcagaaatgtggtatggcttatcaaaaagaacagtgcatacccaacaataaagaggagctacaataataccaaccaagaagatcttttggtactgtggggggttcaccatcctaatgatgcggcagagcagacaaagctctatcaaaatccaaccacctacatttccgttggaacatcaacactgaaccagagattggttccagaaatagctactagacccaaagtaaacgggcaaagtggaagaatggagttcttctggacaattttaaagccgaatgatgccatcaatttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagtatgccattccacaacatacaccccctcaccatcggggaatgccccaaatatgtgaaatcaaacagattagtccttgcgactggactcagaaatacccctcaaagagagagaagaagaaaaaagagaggactatttggagctatagcaggttttatagagggaggatggcagggaatggtagatggttggtatgggtaccaccatagcaatgagcaggggagttgctactctgcagacaaagaatccactcaaaaggcaatagatggagtcaccaataaggtcaactcgatcattaacaaaatgaacactcagtttgaggccgttggaagggaatttaataacttggaaaggaggatagagaatttaaacaagaagatggaagacggattcctagatgtctggacttacaatgctgaacttctggttctcatggaaaatgagagaactctcgactttcatgactcaaatgtcaagaacctttacgacaaggtccgactacagcttagggataatgcaaaggagctgggtaatggttgtttcgaattctatcacaaatgtgataatgaatgtatggaaagtgtaaaaaacggaacgtatgactacccgcagtattcagaagaagcaagactaaacagagaggaaataagtggagtaaaattggaatcaatgggaacttaccaaatactgtcaatttattcaacagtggcgagttccctagcactggcaatcatggtagctggtctatctttatggatgtgctccaatggatcgttacaatgcagaatttgcatttaa

SEQ ID NO: 11 - DNA, 1704 bp, Influenza A virus (A/Anhui/1/2005)
atggagaaaatagtgcttcttcttgcaatagtcagccttgttaaaagtgatcagatttgcattggttaccatgcaaacaactcgacagagcaggttgacacaataatggaaaagaacgttactgttacacatgcccaagacatactggaaaagacacacaacgggaagctctgcgatctagatggagtgaagcctctgattttaagagattgtagtgtagctggatggctcctcggaaacccaatgtgtgacgaattcatcaatgtgccggaatggtcttacatagtggagaaggccaacccagccaatgacctctgttacccagggaatttcaacgactatgaagaactgaaacacctattgagcagaataaaccattttgagaaaattcagatcatccccaaaagttcttggtccgatcatgaagcctcatcaggggtgagctcagcatgtccataccagggaacgccctcctttttcagaaatgtggtatggcttatcaaaaagaacaatacatacccaacaataaagagaagctacaataataccaaccaggaagatcttttgatactgtgggggattcatcattctaatgatgcggcagagcagacaaagctctatcaaaacccaaccacctatatttccgttgggacatcaacactaaaccagagattggtaccaaaaatagctactagatccaaagtaaacgggcaaagtggaaggatggatttcttctggacaattttaaaaccgaatgatgcaatcaacttcgagagtaatggaaatttcattgctccagaatatgcatacaaaattgtcaagaaaggggactcagcaattgttaaaagtgaagtggaatatggtaactgcaacacaaagtgtcaaactccaataggggcgataaactctagtatgccattccacaacatacaccctctcaccatcggggaatgccccaaatatgtgaaatcaaacaaattagtccttgcgactgggctcagaaatagtcctctaagagaaagaagaagaaaaagaggactatttggagctatagcagggtttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaataacttagaaaggagaatagagaatttaaacaagaaaatggaagacggattcctagatgtctggacttataatgctgaacttctggttctcatggaaaatgagagaactctagacttccatgattcaaatgtcaagaacctttacgacaaggtccgactacagcttagggataatgcaaaggagctgggtaacggttgtttcgagttctatcacaaatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagttgcgagttctctagcactggcaatcatggtggctggtctatctttgtggatgtgctccaatgggtcgttacaatgcagaatttgcatttaa

SEQ ID NO: 12 - DNA, 1707 bp, Influenza A virus (A/turkey/Turkey/1/05)
atggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaaggagagagaagaagaaaaaagagaggactatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaa

SEQ ID NO: 13 - protein, 8 aa, Influenza virus HA polybasic cleavage site
REGRRRKR

SEQ ID NO: 14 - protein, 566 aa, Influenza virus A/California/04/2009
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 15 - protein, 566 aa, Influenza virus A/Chile/l/1983
MKAKLLVLLCALSATDADTICIGYHANNSTDTVDTVLEKNVTVTHSVNLLEDNHNGKLCKLKGIAPLQLGKCSIAGWILGNPECESLFSKKSWSYIAETPNSENGTCYPGYFADYEELREQLSSVSSFERFEIFPKESSWPKHNVTKGVTAACSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSIIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI